(19) United States
(12) Patent Application Publication
(10) Pub. No.: US 2003/0130622 A1
(43) Pub. Date: Jul. 10, 2003

(54) Simply and easily used safety syringe cap

(76) Inventor: Spencer Tsai, Taipei (TW)

Correspondence Address: Spencer Tsai, 235 Chung-Ho, Box 8-24, Taipei, TW

(21) Appl. No.: 10/042,454

(22) Filed: Jan. 10, 2002

(51) Int. Cl. A61M 5/32

(52) U.S. Cl. 604/192

(57) ABSTRACT

A simply and easily used safety syringe cap comprises a first cap, a second cap overlapped on the first cap. The first cap serves for sealing a syringe before being used so as to protect the syringe needle. At an opening of the second cap is formed with a trench along a longitudinal direction of the cap. Two sides of the trench are folded outwards to form the sliding pieces. Thereby, after the syringe needle is used, it may be guided into the second cap along the lateral directions of the sliding pieces. The trench is gradually reduced along the opening of the second cap. Thereby, the syringe needle can be guided into the cap by its lateral side to avoid hurting the user.

### FIELD OF THE INVENTION

The present invention relates to articles and instruments for medical use, and particularly to a syringe cap which including a first cap and a second cap, in that a syringe needle can be guided into the cap along sliding pieces.

### BACKGROUND OF THE INVENTION

It is possible that a used syringe needle prinks the medical personnel due to a careless operation. However, this is dangerous, since it is possible that the syringe needle is infected by germs or virus, especially, AIDS, hepatitis, etc. If the syringe needle with these germs or viruses is prinked into a human body, it will induce that the body is infected.

Therefore, some safety syringe needles are developed for improving this defect. However, most of these designs are expensive and has a complicate structure. The complexity of the structure will induce the parts of the syringe needle are tightly engaged and thus cannot work normally. Furthermore, the syringe needle will be destroyed after being used once and thus cannot be used repeatedly. Therefore, this prior art design does not be used widely.

### SUMMARY OF THE INVENTION

Accordingly, the primary object of the present invention is to provide a simply and easily used safety syringe cap comprises a first cap, and a second cap overlapped on the first cap. The first cap serves for sealing a syringe before being used so as to protect the syringe needle. At an opening of the second cap is formed with a trench along a longitudinal direction of the cap. Two sides of the trench are folded outwards to form the sliding pieces. Thereby, after the syringe needle is used, it may be guided into the second cap along the lateral directions of the sliding pieces. The trench is gradually reduced along the opening of the second cap. Thereby, the syringe needle can be guided into the cap by its lateral side so as to avoid hurting the user.

Another object of the present invention is to provide a simply and easily used safety syringe cap, wherein two outer folded sliding pieces from two sides of the trench of the second cap are extended horizontally as two wings.

A further object of the present invention is to provide a simply and easily used safety syringe cap, wherein two inner lateral sides of the trench of the second cap are extended with elastic shielding pieces so that after the syringe needle is guided into the trench, the shielding pieces have functions of opening and closing as a door.

The various objects and advantages of the present invention will be more readily understood from the following detailed description when read in conjunction with the appended drawing.

### BRIEF DESCRIPTION OF THE DRAWINGS

FIG. 1 is a perspective view showing the syringe and cap of the present invention.

FIG. 2 shows the first operation about guiding a syringe needle into the second cap of the present invention.

FIG. 3 shows the second operation about guiding a syringe needle into the second cap of the present invention.

FIG. 4 shows that a syringe needle has been guided into the second cap of the present invention.

FIG. 5 shows that the syringe needle is guided into the second cap.

FIG. 6 shows another embodiment of the syringe cap of the present invention.

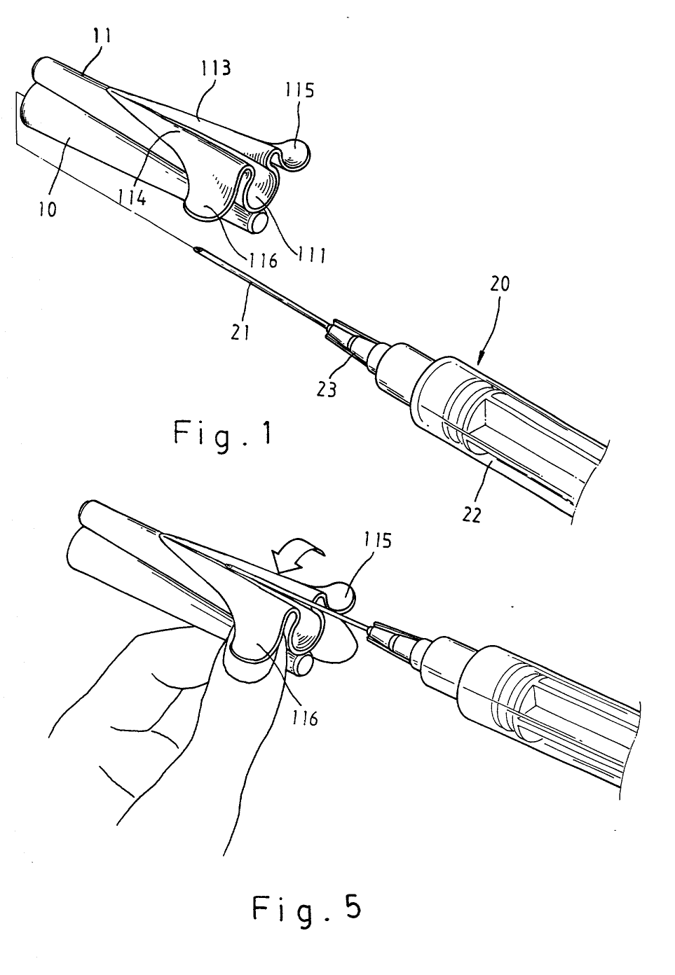

### DETAILED DESCRIPTION OF THE PREFERRED EMBODIMENTS

Referring to appended figures, the safety syringe cap of the present invention is illustrated. The safety syringe cap includes a first cap 10, a second cap 11 overlapping the first cap 10. The first cap 10 is like a prior art syringe cap for sealing a syringe before being used so as to protect the syringe needle 21. At the opening 111 of the second cap 11 is formed with a gradual reduced trench 112 along the longitudinal direction of the cap, two sides of the trench 112 are folded outwards to form the sliding pieces 113, 114. Thereby, after the syringe needle 21 is used, it may be guided into the second cap 11 along the lateral directions of the sliding pieces 113, 114.

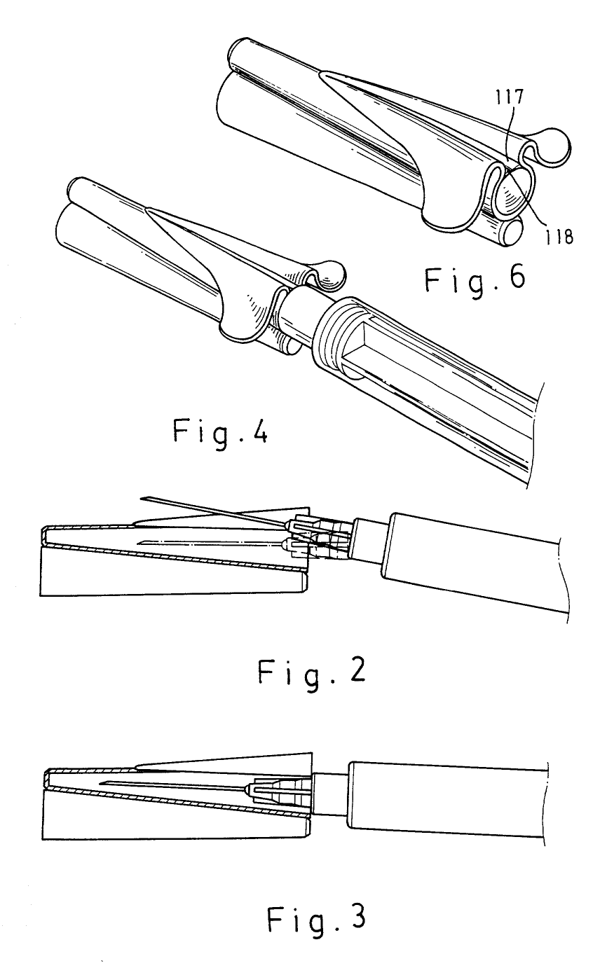

Referring to FIGS. 2 to 4, a pivotal portion is formed between the syringe needle 21 and a cylinder 22. By the guiding of the sliding pieces 113, 114, the syringe needle 21 can slide into the trench 112 of the second cap 11 so as to be shielded therein. Then, it is transferred for being further processed so as to control the cost.

Referring to FIG. 5, in the present invention, two outer folded sliding pieces 113, 114 from two sides of the trench 112 of the second cap 11 can be extended horizontally as two wings 115 and 116 which have the functions of shielding and hindering, as the embodiment illustrated in the drawing. Thereby, as the syringe needle 21 is guided into the trench 112, it will not hurt the fingers of the user.

Referring to FIG. 6, the two inner lateral sides of the trench 112 of the second cap 11 are extended with elastic shielding pieces 117, 118 so that after the syringe needle 21 is guided into the trench 112, the shielding pieces 117, 118 have functions of opening and closing like a door for preventing the syringe needle 21 from releasing along an opposite direction after the syringe needle 21 is engaged with the cap 11.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A simply and easily used safety syringe cap comprising:
a first cap for sealing a syringe before being used so as to protect the syringe needle; and
a second cap overlapped on the first cap; at an opening of the second cap being formed with a trench along a longitudinal direction of the cap, two sides of the trench being folded outwards to be formed with two sliding pieces;
wherein, after the syringe needle is used, it is guided into the second cap along the lateral directions of the sliding pieces.

2. The simply and easily used safety syringe cap as claim in claim 1, wherein the trench is gradually reduced along the opening of the second cap.

3. The simply and easily used safety syringe cap as claim in claim 1, wherein two outer folded sliding pieces from two sides of the trench of the second cap are be extended horizontally and inwards as two wings.

4. The simply and easily used safety syringe cap as claim in claim 1, wherein two inner lateral sides of the trench of the second cap are extended with elastic shielding pieces so that after the syringe needle is guided into the trench, the shielding pieces have functions of opening and closing like a door, thereby, the syringe will not retract backwards.
